(19) United States
(12) Patent Application Publication
(10) Pub. No.: US 2019/0198175 A1
(43) Pub. Date: Jun. 27, 2019

(54) METHOD AND MOBILE APPLICATION FOR OPTIMIZING INFANT FEEDING PLAN

(71) Applicant: MEDELA HOLDING AG, Baar (CH)

(72) Inventor: Ron Sallade, Palatine, IL (US)

(21) Appl. No.: 16/176,150

(22) Filed: Oct. 31, 2018

Related U.S. Application Data
(60) Provisional application No. 62/585,135, filed on Nov. 13, 2017.

(51) Int. Cl. G16H 50/30; G16H 20/60

(52) U.S. Cl. CPC G16H 50/30; G16H 20/60

(57) ABSTRACT

A system configured to analyze targeted breastmilk prediction levels is set forth. The system includes a first data input of formation related to the mother's health. A second data input of information related to a baby's health and the first data provide inputs to a processor in the system for analyzing the data to generate a first output determining first breastpumping activity to reach an initiation target. Additional data input, analyzed by the processor, is analyzed and updates at least one of the initiation target information, build the breastmilk supply, and maintain the breastmilk supply, to generate an updated output for the system.

### CROSS-REFERENCE TO RELATED APPLICATIONS

This patent application claims priority to U.S. Provisional Patent Application No. 62/585,135 filed Nov. 13, 2017, entitled “Method and Mobile Application for Optimizing Infant Feeding Plan,” the entire disclosure of which is hereby incorporated by reference.

### FIELD OF DISCLOSURE

The present disclosure relates generally to a method and mobile application for optimizing and updating an infant feeding plan. The method and mobile application analyze various input data concerning both a mother and her infant, such as goals, preferences, risks, and updates to provide an appropriate feeding plan at any given time. The mobile application helps both mothers and connected medical caregivers improve the management of infant feeding based on improved, proactive care directed to both mother and baby.

### BACKGROUND

Mobile applications that assist mothers with tracking breastmilk volume production are available at various app stores. Such applications typically assist the mother by providing information to help the mother match the volume of breastmilk presently used by the infant. However, presently used volume does not inform the mother as to the volume that might be needed at a later date, such as, for example, upon discharge from the NICU or when a disruption to the mother's supply occurs. Additionally, the mother's preferences and health concerns and risks of mother and baby are not factored into an appropriate plan for a particular infant and mother.

Other publically available information regarding breast feeding and/or pumping, information for babies is currently available. One example is “Your Guide to Breastfeeding” available online from the U.S. Department of Health and Human Services, Office on Women's Health at the following link: https://www.womenshealth.gov/publications/our-publications/breastfeeding-guide/breastfeedingguide-general-english.pdf. (See page 29, column 2).

The aforementioned publication recommends that mothers express milk as often as they would have breastfed an infant who is not yet ready to breastfeed, “ . . . about 8 times in a 24-hour period.” However, pumping or expressing to meet current supply needs fails to prepare the mothers to initiate and build a milk supply that can maintain future supply needs for certain infants, such as premature infants, or under certain adverse conditions, such as an absence or unexpected illness for the mother.

In addition, there are no known tools or digital platforms that automatically bridge relevant health data of the mother and her baby after birth with a medical professional to generate a custom infant feeding plan accessible by both the mother and the medical professional.

### SUMMARY

In accordance with the principles of the present disclosure, a system configured to improve proficiency regarding infant feeding for the mother, reduce lactation risks, and builds a custom feeding plan for the mother's infant are set forth. The system can analyze inputs regarding the health of the mother and baby, as well as of provide an initiation target output to guide the frequency and duration of breast pumping sessions. The initiation target output can be updated as needed. The system can further generate a customized infant feeding plan that allows the mother and medical professional to share real-time information and updates that alter the customized feeding plan. Components of the system can be configured to receive current breast milk output per session data, and analyze the current breast milk output per session data in view of weight information data to form the initiation target output. Additionally, the system can generate warning notifications based on changes in the current breast milk output per session data and/or the weight information data.

The system can be configured so that the initiation target output is updated as needed each time current breast milk output per session data is entered into the system. The weight information data can include any relevant medical information that impacts feeding demand reflected in the initiation target output, such as number of days since birth, current baby weight information, expected baby weight at discharge information, added breast milk production recommended due to health prognosis of the baby or mother impacting feeding, added breast milk production recommended due to medication impact on weight gain, initiation target output norms and other weight validating factors such as bone mass, to name a few. The system can be further configured to store, locally or remotely, or transmit the initiation target output.

The system can be configured so that the initiation target output data can be transmitted to a pumping database. The pumping database can be configured to update a program operably connected thereto in order to generate normative statistics contained in either the weight information data or in the current breast milk output per session data contained in a received initiation target output data, and to automatically update normative statistics contained in the pumping database to adjust initiation target output norms based on the received initiation target output data to better help mothers meet their baby's needs during transition at hospital discharge.

In addition, the system can be configured to give mothers visibility to remote breast milk inventory stored at a medical facility in real time. The system can import breast milk inventory information and add the information to the current breast milk output per session data. The system can then update the initiation target output data to include both current breast milk supply and output information. The goals of the mother can further be included in the update to the initiation target output data.

Various advantages of the present disclosure are specifically described below in reference to the exemplary embodiments, or conceptually embodied therein. The drawings and description herein are provided to merely illustrate examples of the general concepts discussed throughout the present disclosure. Numerous changes and modifications can be made, as known to those of skill in the art, without departing from the general principles set forth herein. In addition, all patents and publications referenced are incorporated herein by reference in the entirety.

### BRIEF DESCRIPTION OF THE DRAWINGS

These and other features and advantages of the various exemplary embodiments disclosed herein will be better understood with respect to the following description and drawings, in which:

FIG. 1 is a flow chart of a method constructed in accordance with the principles herein;

FIG. 2 is a schematic view of an exemplary system constructed in accordance with the principles herein;

FIG. 3 is an exemplary interface of a suitable device for inputting and displaying information from a user, hospital, or doctor's office, and for communicating with a medical interface of a system constructed in accordance with the principles herein;

FIG. 4 is an exemplary interface of a suitable medical interface of a system constructed in accordance with the principles herein;

FIGS. 5A, 5B, 5C, and 5D illustrate exemplary embodiments of various input data regarding a customized feeding plan, risk factors, birth details, and proficiency information, respectively, that can be stored, updated and analyzed in accordance with the principles of the present disclosure; and

FIG. 6 illustrates an exemplary interface panel showing various entity data that can provide measurements of medical performance regarding the dashboard metrics.

Common reference numerals are used throughout the drawings and the detailed description to indicate the same elements.

### DETAILED DESCRIPTION

The detailed description set forth below in connection with the appended drawings is intended as a description of certain exemplary embodiments of various system components constructed in accordance with the principles herein, including. These examples are not intended to represent the only embodiments or forms that may be developed or utilized according to these principles. It is further understood that the use of relational terms such as first and second, and the like are used solely to distinguish one entity from another without necessarily requiring or implying any actual such relationship or order between such entities.

In certain medical situations, the increase in breastmilk demanded by an infant does not happening in the normal physiologic window for lactation. These situations can include premature birth, and changes in the health of a mother or baby that adversely affect milk supply. For premature babies, mothers must initiate the milk supply to a higher capacity soon after birth to compensate for the lag in physiologic response. For much of the very low birth weight (VLBW) neonatal intensive care unit (NICU) stay, 200-300 ml of breastmilk may be sufficient to feed the baby, but 500 ml+ might be required to build the breastmilk supply for the mother to attain exclusive human milk feeding at hospital discharge.

Certain aspects of some of the exemplary embodiments constructed in accordance with the principles herein are directed toward a system that provides a database of pumping volumes and more importantly, feeding volumes, that can inform a predictive model for a confidence interval on feeding volume at a future gestational age (discharge). The database may use information such as gestational age at birth, weight, sex, and feeding volume history. Information could be collected through connected Medela enteral feeding (EF) pumps and/or warmers, breast pumps, hospital records, as well as direct entry by a mother or caregiver. Further, milk inventory, such as in the NICU, can be converted and displayed in days of current feeding or future feeding volume (i.e. at discharge). Standard NICU feeding progression protocols can inform near future demand. In accordance with the principles herein, the predictive model could inform the mother and the healthcare team of needed volumes closer to hospital discharge. The predictive model can be repeatedly compared to mothers supply information to update and inform the mother of the need for higher milk production in the early days of lactogenesis, or at any time during her breast pumping journey, where interventions may be more effective.

As will be described in more detail below, several embodiments are contemplated in accordance with the principles herein.

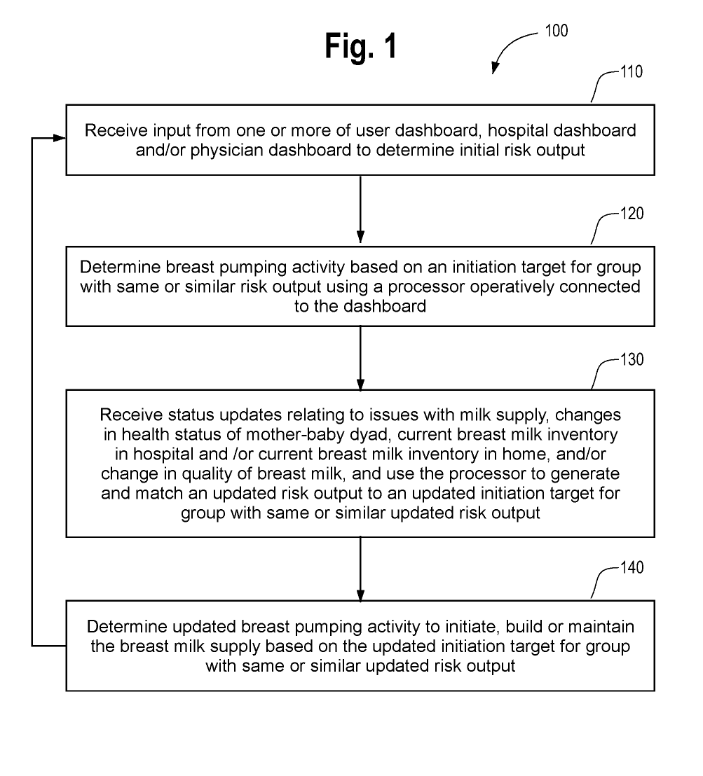

An exemplary embodiment of a method constructed in accordance with the principles herein is shown generally at 100 in FIG. 1. In accordance with the principles herein, an exemplary description of a mother's lactogenesis or breast milk creation is provided below. The exemplary description is constructed in accordance with the principles herein and can provide the underlying information needed for one embodiment of a predictive breastmilk supply system.

### Example Lactogenesis Journey of Mother—Breast Milk Creation

Generally, there are four phases of production capability of milk from a mother. The four phases include a develop phase, an initiate phase, a build phase, and a maintain phase. Typically, there is no production of milk from the mother during the develop phase. In the initiate phase, a low level of milk is first produced by the mother and then increases. The level of milk produced by the mother then further increases after the initiate stage and into the build stage. It is desired to maintain the highest level of milk produced possible in the maintain phase. Each of the phases includes a number of days or amount of time, for example, which varies depending upon the mother and birthweight of the baby. In one example, a 1500 g VLBW preemie may achieve about 260 mL of feeding volume by day 11, compared to milk production that should be over 500 mL at day 11 source: UCSD iEAT feed advancement protocol). In contrast, term infants ramp quickly to feed volumes consistent with adequate milk production in the first 11 days (average 654, Neville, et al.). If moms set targets for Preemie demand they may have insufficient supply at the time of infant discharge.

The method shown generally at 100 in FIG. 1 includes the first step 110 of determining an initial risk output by analyzing input data regarding a mother and her baby transmitted via a suitable device of a system, such as a user dashboard, hospital dashboard, physician dashboard or the like. The input data can include details regarding the health and associated risks for both the mother and infant. At step 120, breast pump activity is determined by mapping an initiation target to a group with the same or similar risk output from step 110 using a processor operatively connected to the dashboard. Additional status updates are input into the system 100 updating the output with additional data and analysis relating to at least one of health status, breast milk inventory at any location, change in quality of breast milk, issues with milk supply, or other factors at step 130. Updated breast pumping activity is then determined at step 140 based on a group or data update that results in a change to the breast pumping activity. The breast pumping activity can be based on a customized infant feeding plan or on a combination of factors, such as the mother's goals and the customized feeding plan. In this way the updates to the breast pumping activity help reduce the mothers chance of suppressed lactation, and combined with improved mother proficiency, improve the mother's chances of meeting her goals. Moreover, the nursing staff caring for the infant can effectively interact with the mother regarding the infant feeding plan and assist her with proactive care when needed. In this way the nurses can match the right care to the right mother at the right time to improve the chances of success for the infant feeding plan.

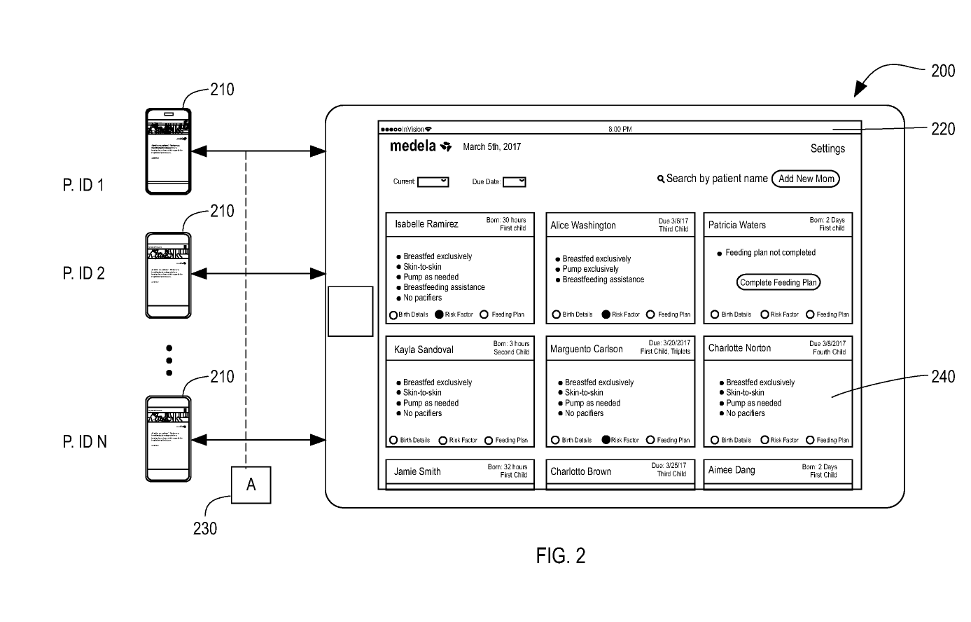

An exemplary embodiment of a system constructed in accordance with the principles herein is shown generally at 200 in FIG. 2. The system 200 includes suitable input devices 210 configured for operative communication to a medical interface 220, which can also be employed to input data to the system. The devices 210 and 220 can be used to input any relevant data that can affect breast milk generation, such as data regarding the mother's health, baby's health or additional data such as health risk factors. Suitable devices for inputting the data can include computing devices, mobile devices, tablets, smartphones, and the like. A single device can be used to input the data into the system. A processor 230 in the system receives and analyzes the data to generate exemplary output 240 that informs the mother and/or the healthcare team of the needed breast pumping schedule via a suitable display, audio output, electronic notice, or other output conveying device, to help the mother reach the initiation target or to build or maintain the breastmilk supply.

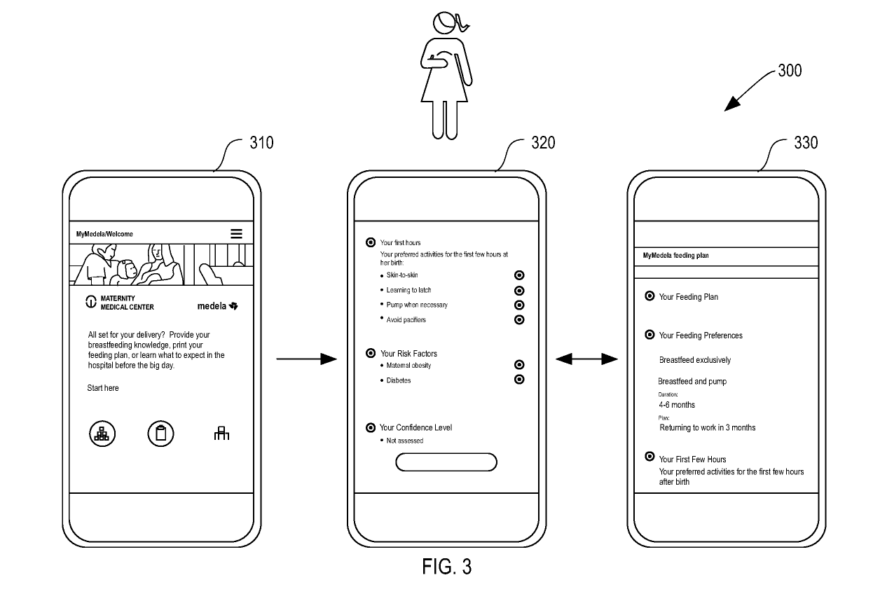

As illustrated in FIG. 3, a suitable user or medical interface can include a display 310 that indicates the medical center managing the data, and provides links to education, a feeding plan, and information regarding the hospital experience. Another interface 320 can display data input buttons that help customize the feeding plan for an infant based on the preferences and risk factors input. Additional information can augment the risk and plan information as well. The information stored and generated in the system 300 can be transferred to an app at any time, such as the MyMedela App at 330.

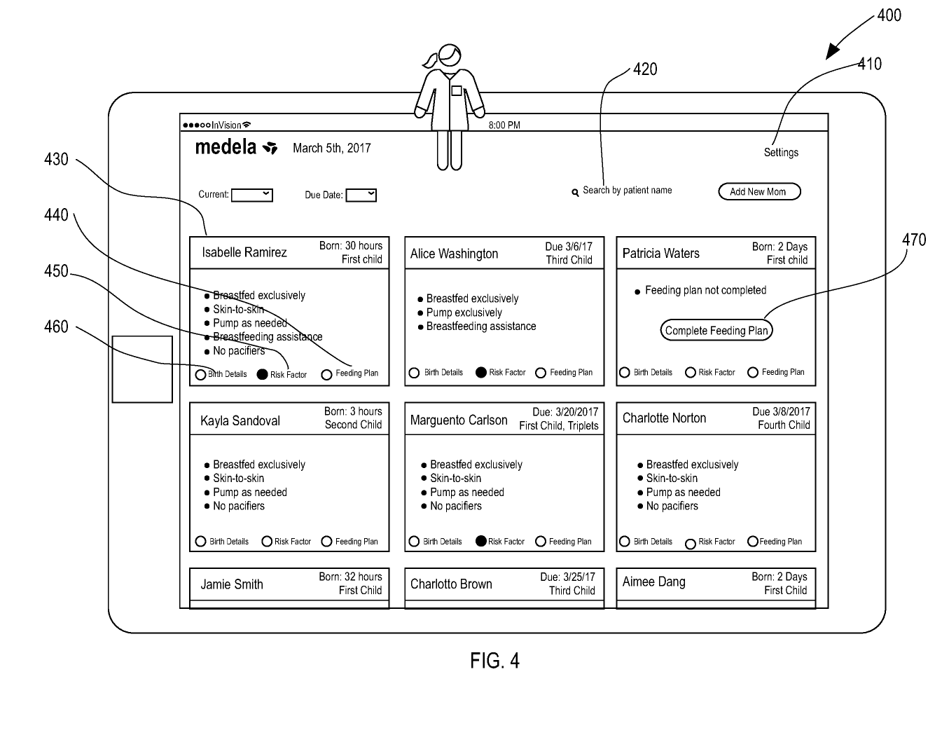

As illustrated in FIG. 4, a system 400 can provide a medical interface, or nurse dashboard, 410 that includes settings determined by a hospital. The mother's input into the initiation digital health service application on her smartphone can populate automatically in the nurse dashboard. Alternatively, a medical provider can input the information visible on the nurse dashboard. The interface 410 can be configured to facilitate a search patient by name function at 420, or a hospital ID or other identifier can be used in place of a name. An individual patient record 430 can be sorted by mother or baby name. The patient record 430 can include a feeding plan tab 440, risk factors tab 450, birth details tab 460, time since birth, and can indicate the mother preferences and child's birth order. Other parameters relevant to the heath and risk factors of the mother and child can be included as well, such as the mother's proficiency, which can be estimated based on input from the mother and/or training provided within the application. Additionally, dynamic milk inventory information from any location can be stored in the record or provided in a tab within the interface display. If the input has not yet been completed regarding the feeding plan, a notification 470 can be displayed to indicate that information is needed to proceed.

A system constructed in accordance with the principles herein can continually update and generate suitable feeding plans for any infant, including hospitalized and/or at risk infants. The system can also inform and encourage the mother regarding her progress. As use of the application continues, the data becomes more refined and provides more definite predictive outcomes with increased group data for groups with same or similar risk output.

Customizable feeding plans generated by applications constructed in accordance with the principles herein allow professional medical care providers to assess the percentage of patients that initiate breast pumping and/or breast feeding, the level of patient satisfaction, and alignment among staff on feeding goals. Since a critical window starts one to three hours after birth for mothers wanting to breast feed their child, appropriate use of risk mitigating digital tools can increase the chance of success for mother's wanting to breast feed their infants. Additionally, customized feeding plans derived from systems configured in accordance with the principles of the present disclosure result in feeding plans that are consistent with nursing care. Nurses can proactively use technology to assist with the feeding plans, and improve both patient satisfaction and outcomes. As a result, the mother's expectations and the nurses care approach can both be bridged by the mobile application, and can lead to greater success regarding the updated customized feeding plan of the mother's infant.

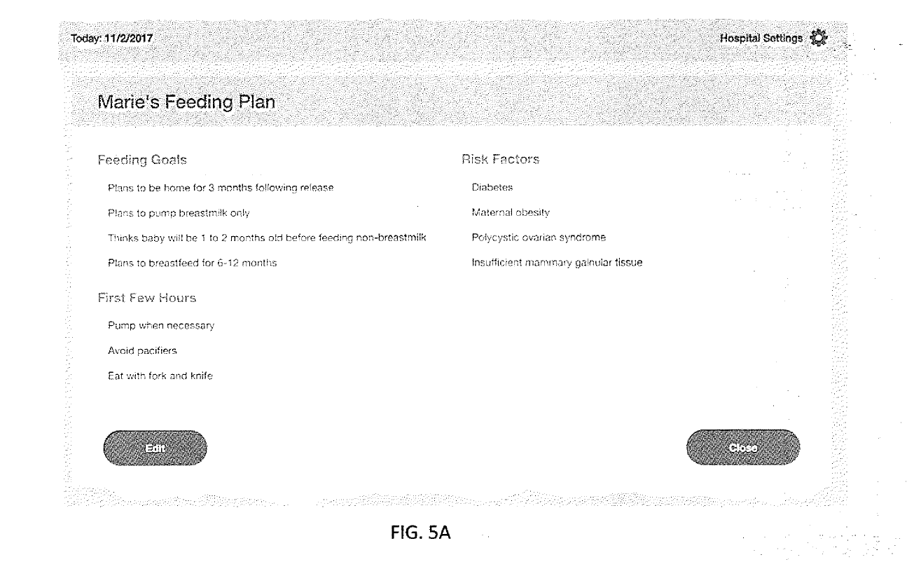

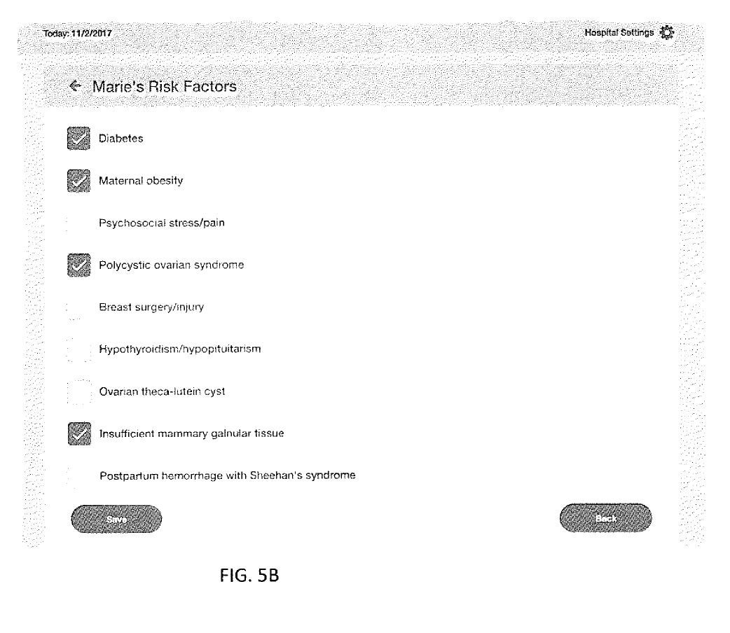

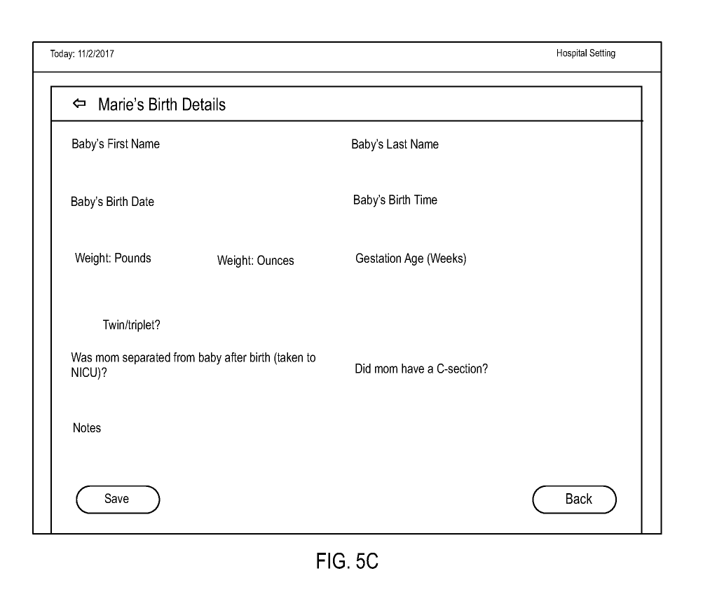

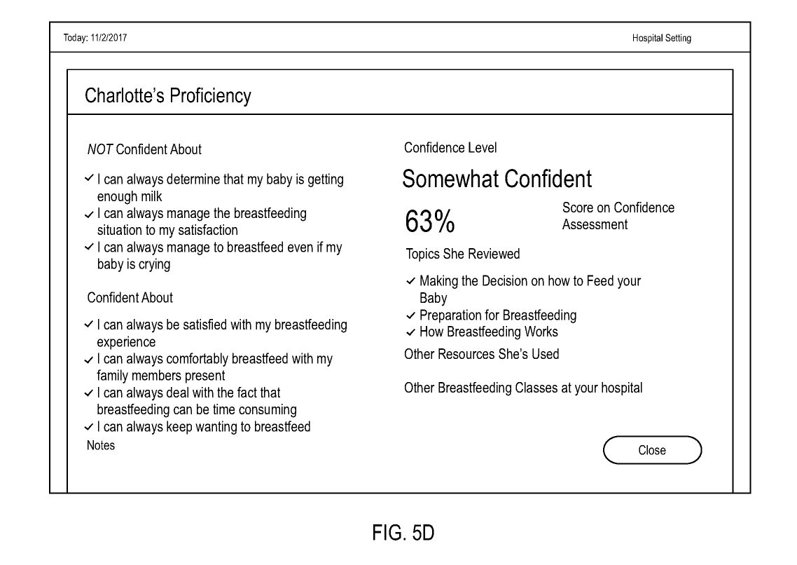

FIGS. 5A, 5B, 5C, and 5D illustrates exemplary embodiments of various input data regarding a customized feeding plan, risk factors, birth details, and proficiency information, respectively, that can be stored, updated and analyzed in accordance with the principles of the present disclosure.

As shown in FIG. 5B, lactation risk factors can include, for example, the presence of certain health conditions and/or risks of the mother. Such risks can include, for example, diabetes, maternal obesity, psychological stress and/or pain, polycystic ovarian syndrome, breast surgery/injury, hypothyroidism/hypopituitarism, ovarian theca-lutein cyst, insufficient mammary glandular tissue, postpartum hemorrhage with Sheehan's syndrome, and any other health risk or condition that could increase the lactation risk for the mother.

As shown in FIG. 5C, lactation risks factors can further include, for example, the presence of certain health conditions and/or risks of the baby. Such factors can include the weight of the baby at birth, gestational age at birth, type of birth, separation at birth, whether a twin or triplet or other multiple birth baby, as well as specific health challenges for the baby, or any other factor that increases the lactation risk for the mother.

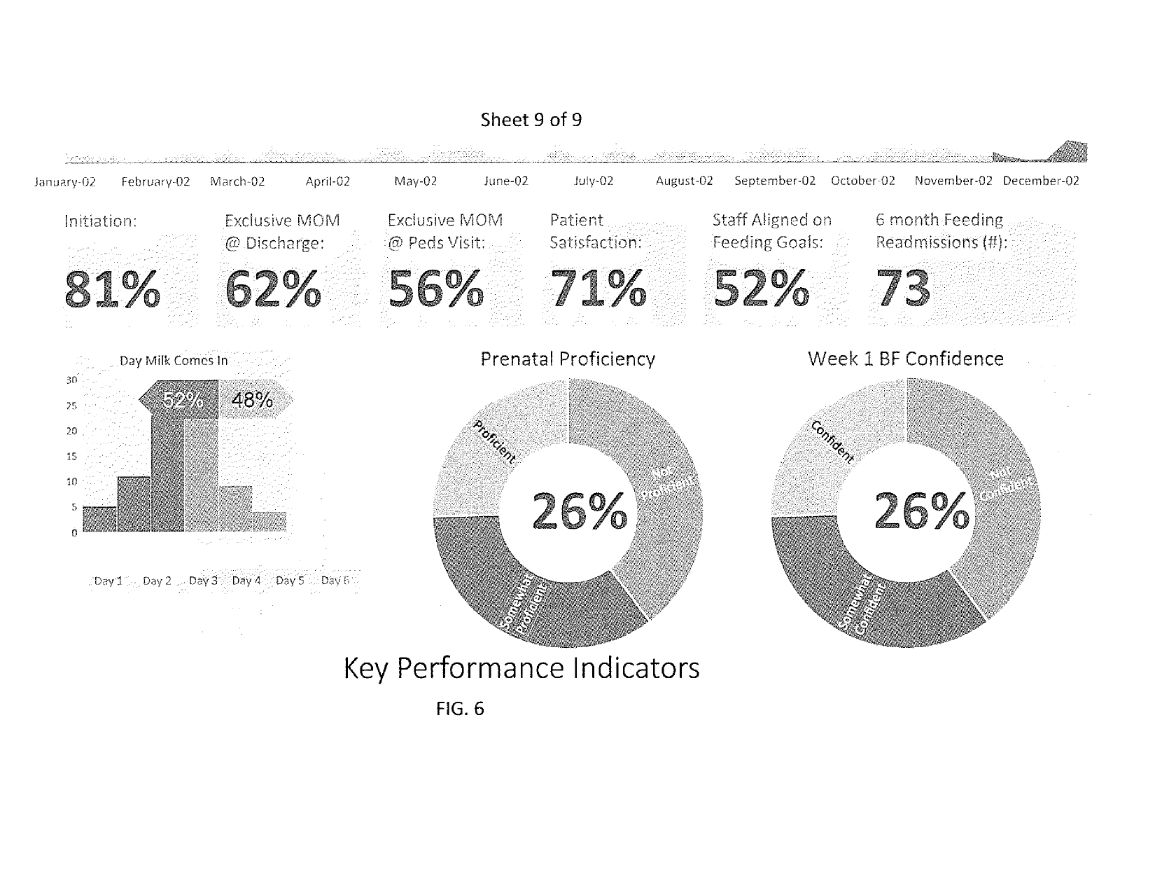

FIG. 6 illustrates various entity data that can provide measurements of medical performance regarding the dashboard metrics. The dashboard metrics over time allow the entity to access more clearly the influence of medical care on the success of the feeding plan. The entity can review factors, such as how many mothers are exclusively breastfeeding. The data can be collected during the time the care is rendered, rather than in a post care survey as is typically done. All moms can be assessed by an entity, and clinicians can optimize the opportunity for mom to meet her feeding needs.

Variations of the specific device configurations shown and described herein that provide a are within the scope of the principles of the present disclosure, and are included in all claims deriving therefrom.

What is claimed:

1. A system configured to analyze targeted breastmilk prediction levels comprising:
a first data input of formation related to the mother's health; a second data input of information related to a baby's health;
a processor in the system for analyzing the data to generate a first output determining first breastpumping activity to reach an initiation target; and
additional data input analyzed by the processor to at least one of update the initiation target information, build the breastmilk supply, and maintain the breastmilk supply.
